(19) United States
(12) Patent Application Publication
(10) Pub. No.: US 2009/0270840 A1
(43) Pub. Date: Oct. 29, 2009

(54) DELIVERY SYSTEMS FOR A MEDICAL DEVICE AND RELATED METHODS

(75) Inventors: Scott D. Miles, Sandy, UT (US); Richard J. Linder, Sandy, UT (US)

Correspondence Address: Holland & Hart LLP/Coherex Medical, 60 East South Temple, Suite 2000, Salt Lake City, UT, 84111, US

(73) Assignee: COHEREX MEDICAL, INC., Salt Lake City, UT (US)

(21) Appl. No.: 12/413,334

(22) Filed: Mar. 27, 2009

Related U.S. Application Data
(60) Provisional application No. 61/040,608, filed on Mar. 28, 2008.

(51) Int. Cl. A61M 25/01

(52) U.S. Cl. 604/528

(57) ABSTRACT

Delivery systems for a medical device and related methods are provided. In one embodiment the delivery system includes a handle, a handle extension, and a sleeve. The handle extension extends from the handle and includes at least one channel defined therein along a longitudinal length of the handle extension. The handle extension also includes an abutment surface positioned in the at least one channel. The sleeve is disposed around the handle extension and is coupled to a catheter. The sleeve also includes a biased actuator configured to be moved between at least two actuator positions. The sleeve is moveable from a distal position to an intermediate position where it is stopped via the abutment surface to partially unsheathe a medical device from the catheter. The actuator can then be manually actuated to enable displacement of the sleeve to a proximal position to fully unsheathe the medical device from the catheter.

### CROSS-REFERENCE TO RELATED APPLICATIONS

The present application claims the benefit of U.S. Provisional Patent Application Ser. No. 61/040,608, filed Mar. 28, 2008, entitled DELIVERY SYSTEM FOR A MEDICAL DEVICE, the disclosure of which is incorporated by reference herein in its entirety.

### TECHNICAL FIELD

The present invention relates generally to delivery systems for a medical device and, more specifically, to a delivery system for implanting a medical device in a patient and to related methods.

### TECHNOLOGICAL BACKGROUND

Medical devices are utilized on a daily basis in an effort to improve the health and quality of life of various individuals. In many instances, such medical devices include devices or structures that are disposed or implanted within a patient's body. Such devices may be utilized for divergent purposes and may exhibit a any of a number of different configurations.

Some medical devices are even configured to be disposed or implanted within one's heart to repair an identified defect. For example, some devices are utilized to provide an enlarged opening or passage within a blood vessel in order to improve circulation of blood. On the other hand, some devices are utilized to occlude an undesired hole or opening within the heart. In either case, it is desired to improve the function of the heart and the circulatory system by correcting some defect

Devices configured to occlude an opening or hole within the heart are often utilized in conjunction with repairing defects such as atrial septal defects (ASD), ventricular-septal defects (VSD), patent ductus arteriousus (PDA) and patent foramen ovales (PFO). Many devices have been developed in an effort to more effectively correct such defects while also being implanted or disposed within one's heart using processes that are less invasive than conventional surgical techniques and, therefore, facilitate relatively quick recoveries from the procedure by the patient.

Various embodiments of a medical device used, for example, in repairing a PFO is described in U.S. patent application Ser. No. 11/836,123 filed on Aug. 8, 2007, entitled METHODS, SYSTEMS AND DEVICES FOR REDUCING THE SIZE OF AN INTERNAL TISSUE OPENING, the disclosure of which is incorporated by reference herein in its entirety. Delivery of such a device to a patient's heart is conventionally accomplished percutaneously utilizing a catheter type device. However, the increasing complexity of medical devices invites improvements in delivery devices and systems to help ensure that the devices are properly delivered and positioned within a patient's body. Sometimes such delivery includes multiple stages of activities, corresponding with multiple stages of deployment of the medical device. Additionally, it is often desirable to retain the ability to recapture the medical device within the delivery device until the medical device is confirmed as being installed correctly and a medical practitioner is satisfied with its final deployment.

Thus, there is a continuing desire to provide delivery devices, systems and methods which help simply and effectively deliver a medical device to a desired location within a patient's body, while providing flexibility to the medical practitioner in delivering the medical device, and potentially recapturing the medical device, during the procedure.

### BRIEF SUMMARY OF THE INVENTION

The present invention is directed to delivery systems for a medical device and related methods. In one embodiment, a delivery system for a medical device includes a handle, a handle extension coupled with the handle, a sleeve disposed about the handle extension, a catheter coupled to the sleeve, and an actuator associated with the sleeve. The sleeve is moveable from a first position to a second position to partially unsheathe a medical device from a distal tip of the catheter. Upon actuation of the actuator, the sleeve is moveable from the second position to a third position to fully unsheathe a medical device from the distal tip of the catheter.

In accordance with another embodiment, a delivery system includes a handle, and a handle extension coupled with the handle. The handle extension includes at least one channel defined therein and along a longitudinal length of the handle extension. At least one abutment surface is associated with the at least one channel. A sleeve is disposed about the handle extension and a catheter is coupled to the sleeve. An actuator having at least one projection is associated with the sleeve and configured to be displaced between at least a first actuator position and a second actuator position. The sleeve is moveable from a first position to a second position wherein the at least one projection of the actuator abuts the at least one abutment surface and wherein the catheter is displaced to partially unsheathe a medical device from a distal tip of the catheter.

In accordance with another embodiment of the present invention, a method of delivering a medical device is provided. The method includes housing a medical device within a tip portion of a catheter and positioning the tip portion of the catheter at a desired location. The medical device is partially unsheathed by displacing a sleeve coupled with the catheter from a first position relative to a handle to a second position relative to the handle, wherein, at the second position, an actuating assembly engages an abutment surface. The actuator is displaced to disengage the actuator from the abutment surface and the sleeve and catheter are displaced to a third position relative to the handle to fully unsheathe the medical device.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1A is a side view of a delivery system for a medical device, depicting a slider in a distal position, according to one embodiment of the present invention;

FIG. 1B is a side view of a distal tip of a catheter, depicting the medical device in outline within the distal tip to correspond with the distal position of the slider in FIG. 1A, according to the present invention;

FIG. 2A is a side view of the delivery system for the medical device, depicting the slider in an intermediate position, according to an embodiment of the present invention;

FIG. 2B is a side view of the distal tip of the catheter, depicting the medical device partially deployed from the distal tip to correspond with the intermediate position of the slider in FIG. 2A, according to the present invention;

FIG. 3A is a side view of the delivery system for the medical device, depicting the slider in the intermediate position and an actuator in a depressed position, according to an embodiment of the present invention;

FIG. 3B is a side view of the distal tip of the catheter, depicting the medical device partially deployed from the distal tip to correspond with the position of the slider in FIG. 3A, according to the present invention;

FIG. 4A is a side view of the delivery system for the medical device, depicting the slider in a proximal position, according to an embodiment of the present invention;

FIG. 4B is a side view of the distal tip of the catheter, depicting the medical device fully deployed from the distal tip to correspond with the position of the slider in FIG. 4A;

FIGS. 5A and 5B show side and bottom views of a delivery system for a medical device according to an embodiment of the present invention, with the delivery system being in a first state;

FIG. 5C is a partial cross-sectional view of the system shown in FIGS. 5A and 5B as indicated by section lines shown in FIG. 5B;

FIG. 5D is a cross-sectional view of the system shown in FIGS. 5A and 5B as indicated by section lines shown in FIG. 5B;

FIGS. 6A and 6B show side and bottom views of a delivery system for a medical device according to an embodiment of the present invention, with the delivery system being in a second state;

FIG. 6C is a partial cross-sectional view of the system shown in FIGS. 6A and 6B as indicated by section lines shown in FIG. 6B;

FIG. 6D is a cross-sectional view of the system shown in FIGS. 6A and 6B as indicated by section lines shown in FIG. 6B;

FIGS. 7A and 7B show side and bottom views of a delivery system for a medical device according to an embodiment of the present invention, with the delivery system being in a third state;

FIG. 7C is a partial cross-sectional view of the system shown in FIGS. 7A and 7B as indicated by section lines shown in FIG. 5B;

FIG. 7D is a cross-sectional view of the system shown in FIGS. 7A and 7B as indicated by section lines shown in FIG. 7B;

FIGS. 8A and 8B show side and bottom views of a delivery system for a medical device according to an embodiment of the present invention, with the delivery system being in a fourth state;

FIG. 8C is a partial cross-sectional view of the system shown in FIGS. 8A and 8B as indicated by section lines shown in FIG. 8B;

FIG. 8D is a cross-sectional view of the system shown in FIGS. 8A and 8B as indicated by section lines shown in FIG. 8B;

FIGS. 9A and 9B show side and bottom views of a delivery system for a medical device according to an embodiment of the present invention, with the delivery system being in a fifth state;

FIG. 9C is a partial cross-sectional view of the system shown in FIGS. 9A and 9B as indicated by section lines shown in FIG. 9B;

FIG. 9D is a cross-sectional view of the system shown in FIGS. 9A and 9B as indicated by section lines shown in FIG. 9B;

FIG. 10 is a perspective view of a component of the medical delivery system shown in FIGS. 5A-9D;

FIG. 11 is a partial cross-sectional view of another component of the medical delivery system shown in FIGS. 5A-9D;

FIG. 12 is a partial cross-sectional view of yet another component of the medical delivery system shown in FIGS. 5A-9D;

FIG. 13 is a partial cross-sectional view of a further component of the medical delivery system shown in FIGS. 5A-9D as tethered to a medical device; and

FIGS. 14A-14C depict the deployment of a medical device at various stages thereof in accordance with an embodiment of the present invention.

### DETAILED DESCRIPTION OF THE INVENTION

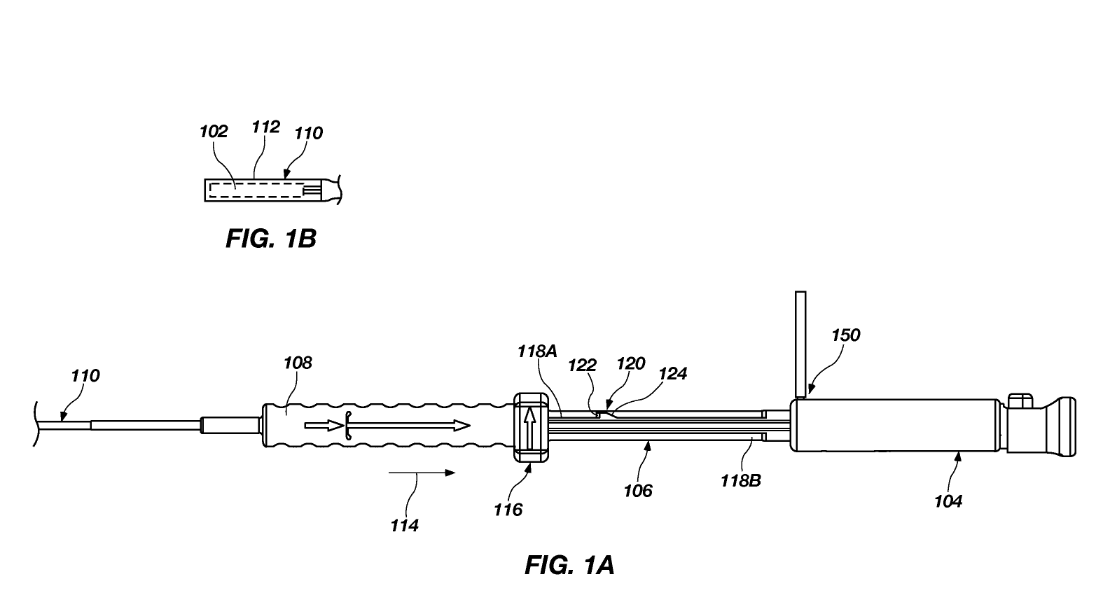

Referring to FIGS. 1A and 1B, a side view of a delivery system 100 is shown. The delivery system 100 is configured to deploy a medical device 102 by unsheathing the medical device 102 in a plurality of stages. For example, in the embodiment shown in FIGS. 1A and 1B, the delivery system 100 is configured to partially unsheathe the medical device 102 in a first stage and then fully unsheathe the medical device 102 in a second stage. Other embodiments may be configured to unsheathe the medical device 102 in additional stages if so desired.

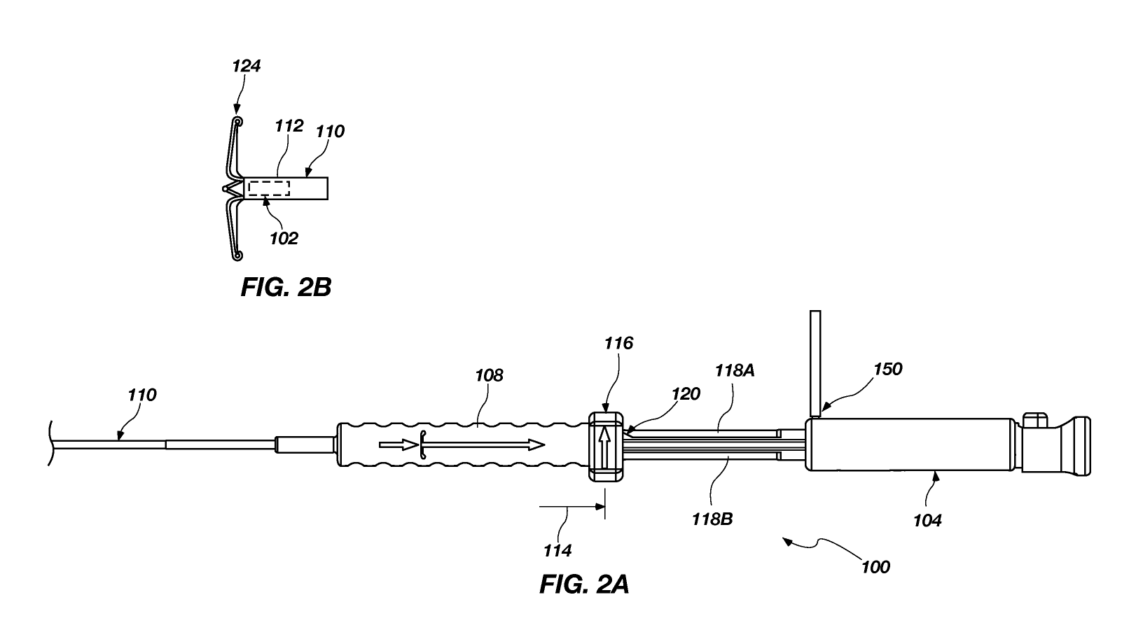

In the embodiment shown in FIGS. 1A and 1B, the delivery system 100 includes a handle 104, a handle extension 106 and a slider or a sleeve 108 that is movable relative to the handle 104. For sake of clarity in the following description, the handle 104 is described as being at the proximal side of the delivery system 100 with the handle extension 106 extending distally from the handle 104. The sleeve 108 is positioned over the handle extension 106 and is coupled to a catheter 110. The medical device 102 is positioned in a distal tip 112 of the catheter 110 and interconnected to the handle 104 via a tether and coil/wire arrangement that extends through the catheter 110. It is noted that the entirety of the catheter 110 is not shown in FIGS. 1A and 1B, but, rather, a significant portion is shown in FIG. 1A while the distal tip 112 is shown in FIG. 1B. (FIGS. 2A, 3A and 4A similarly show a significant portion of the catheter 110, while FIGS. 2B, 3B and 4B show the tip 112 and the medical device 102 in various stages of unsheathing).

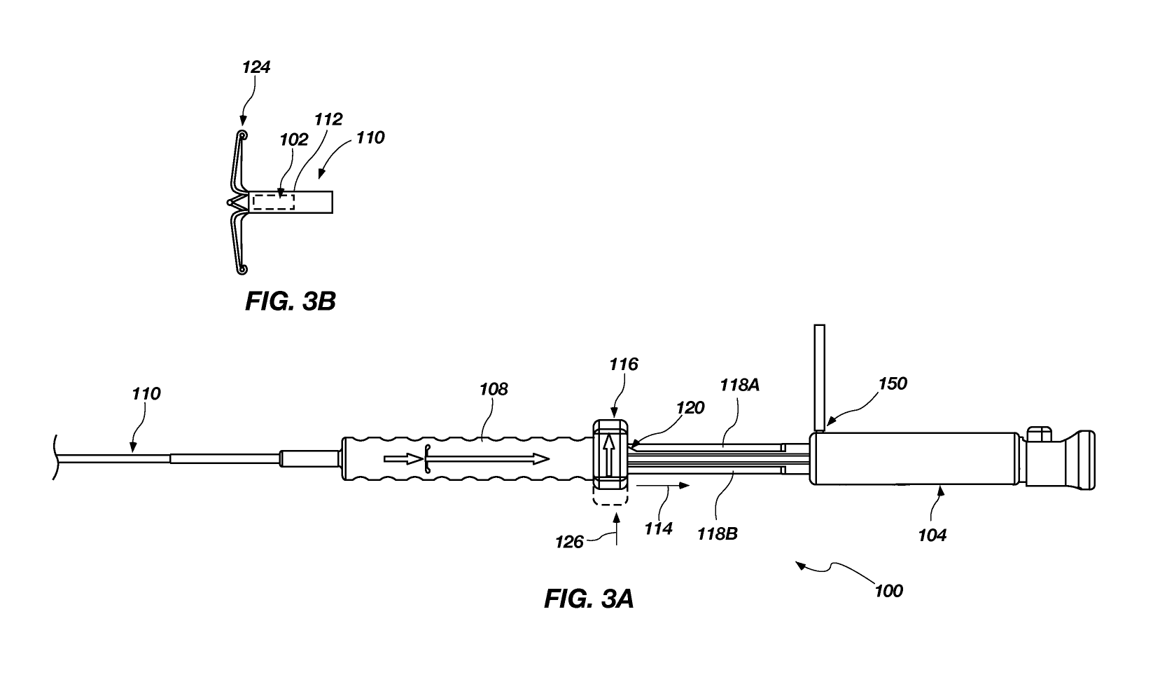

The sleeve 108 is movable, bi-directionally, along the handle extension 106 in a linear manner. From its most distal position (i.e., when it is further from the handle 104), the sleeve 108 is configured to be movable proximally, as indicated by arrow 114, to an intermediate position (e.g., as shown in FIGS. 2A and 2B) to partially unsheathe the medical device 102, after which the sleeve 108 may be moved to its most proximal position to fully unsheathe the medical device 102 (as shown in FIGS. 3A and 3B). As depicted in FIGS. 1A and 1B, the sleeve 108 is in its most distal position with the medical device 102 fully sheathed within the distal tip 112 of the catheter 110.

The sleeve 108 includes a manually operated, spring loaded (or otherwise biased), actuator 116 at a proximal end of the sleeve 108 with two opposing set screws or other projections (not shown) that extend within two corresponding channels 118A and 118B defined on opposite sides of the handle extension 106. Such set screws or projections may extend from the actuator 116 into the channels 118A and 118B at various stages of use of the delivery system 100. One of the channels (i.e., channel 118A) includes a stopper mechanism or structure (referred to herein simply as a stopper 120) configured to stop the sleeve 108 at an intermediate position. Such stopper 120 may include a butt portion 122 (also referred to as an abutment or a shoulder portion) and a tapered portion 124 disposed within the associated channel 118A.

When the sleeve 108 is displaced proximally, the set screw or projection extending into the upper channel 118A will butt against the butt portion 122 of the stopper 120 and stop the sleeve 108 until the actuator 116 is actuated as will be discussed further below. On the other hand, when the sleeve 108 is being displaced distally, from a proximal side of the stopper 120, the set screw or projection disposed within the upper channel 118A will follow the contour of, and be displaced by, the tapered portion 124 of the stopper 120. In other words, when the sleeve 108 is moving from its distal most position to its proximal most position, the stopper 120 acts as a hindrance or a blocking member until the actuator 116 is appropriately actuated. However, the stopper 120 does not act as a hindrance when the sleeve 108 is being displaced from its proximal-most position to its distal-most position.

Now referring to FIGS. 2A and 2B, the sleeve 108 is shown as being displaced proximally from its distal-most position, as depicted by directional arrow 114, until the set screw or projection abuts the butt portion 122 of the stopper 120 (FIG. 1A), thereby preventing it from moving further in the proximal direction. In this position, the catheter 110, which is correspondingly displaced with the sleeve 108, is also moved proximally to partially unsheathe a distal portion 124 of the medical device 102 from the distal tip 112 of the catheter 110.

Referring to FIGS. 3A and 3B, in order to displace the sleeve 108 further in the proximal direction (as compared to that which is shown in FIG. 2A) and fully unsheathe the medical device 102 from the distal tip 122 of the catheter 120, the actuator 116 is first displaced in a direction that is substantially perpendicular to the linear movement of the sleeve 108 (i.e., in a direction as depicted by arrow 126). Movement of the actuator 116 by manual actuation will position the set screw or other projection to a location that is above the butt portion 122 (FIG. 1A) of the stopper 120 so as to enable the sleeve 108 to be further displaced proximally as indicated by directional arrow 114. When this occurs, the other set screw or projection engages the other channel 118B keeping the sleeve 108 aligned with the handle extension 106 as the actuator 116 is displaced beyond the stopper. As previously set forth, the actuator 116 can be spring-loaded or otherwise biased so as to maintain the set screw or other projection within the channel 118A and in the non-actuated position until sufficient external force is applied to the actuator 116 to displace the set screw or projection out of the channel 118A.

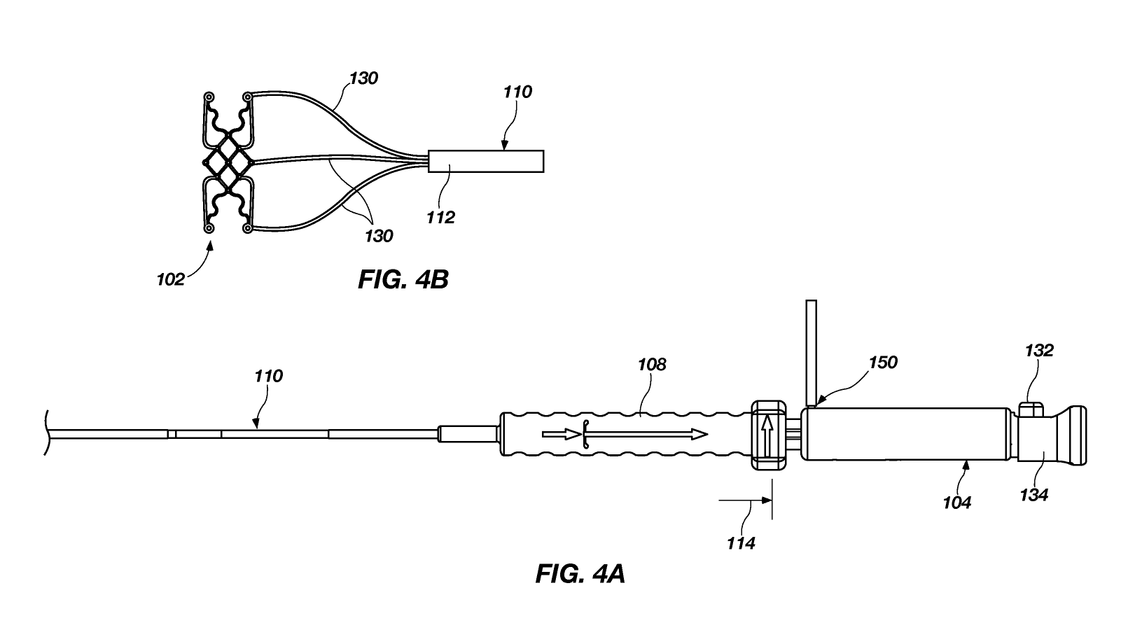

FIGS. 4A and 4B show the sleeve 108 moved to it's most proximal position, as depicted by directional arrow 114. When the sleeve 108 is in this proximal-most position, the catheter 110 is pulled back a predetermined distance relative to the tethers 130 and medical device 102 so as to fully unsheathe the medical device 102 from the distal tip 112 of the catheter 110. If the operator is satisfied with the position of the medical device 102 within the patient, the operator can release the medical device 102 from its associated tethers 130 by depressing a release button 132 at the proximal end of the handle 104 and pulling proximally on the end portion 134 from the handle 104. The tethers 130, being coupled with end portion 134 of the handle 104, withdraw from and release the medical device 102 and the catheter 110 and it's associated components may then be withdrawn from the patient while leaving the medical device 102 in its implanted position.

The delivery system 100 may also include additional components such as, for example, one or more ports 150 for introducing fluid through the catheter 110 as will be appreciated by those of ordinary skill in the art. The components of the delivery system 100 may be formed from a variety of materials and using a variety of manufacturing techniques. For example, components such as the handle 104, extension 106 and sleeve 108 may be formed of appropriate plastic materials suitable for use in a surgical environment. Use of plastic materials enables relatively cost efficient manufacturing of the delivery system 100 as many of the components may be molded. However, other materials, including metals and metal alloys may also be used.

It is also noted that the delivery system may be used with medical devices that are configured differently from that which is shown in FIG. 4B. Other examples, while not limiting, include those described in U.S. patent application Ser. No. 11/836,123. Additionally, as noted above, the delivery system may be configured to accommodate multiple stages of unsheathing. Thus, while only one stage is shown of a partially unsheathed medical device 102, the delivery system may be configured to have multiple stages of a partially unsheathed medical device 102. Moreover, even if the delivery system 100 is configured to have a single stage where the medical device 102 is partially unsheathed, that stage may be tailored to unsheathe more of the medical device 102 than is shown in FIGS. 2B and 3B, or less of the medical device 102 than is shown in such drawings.

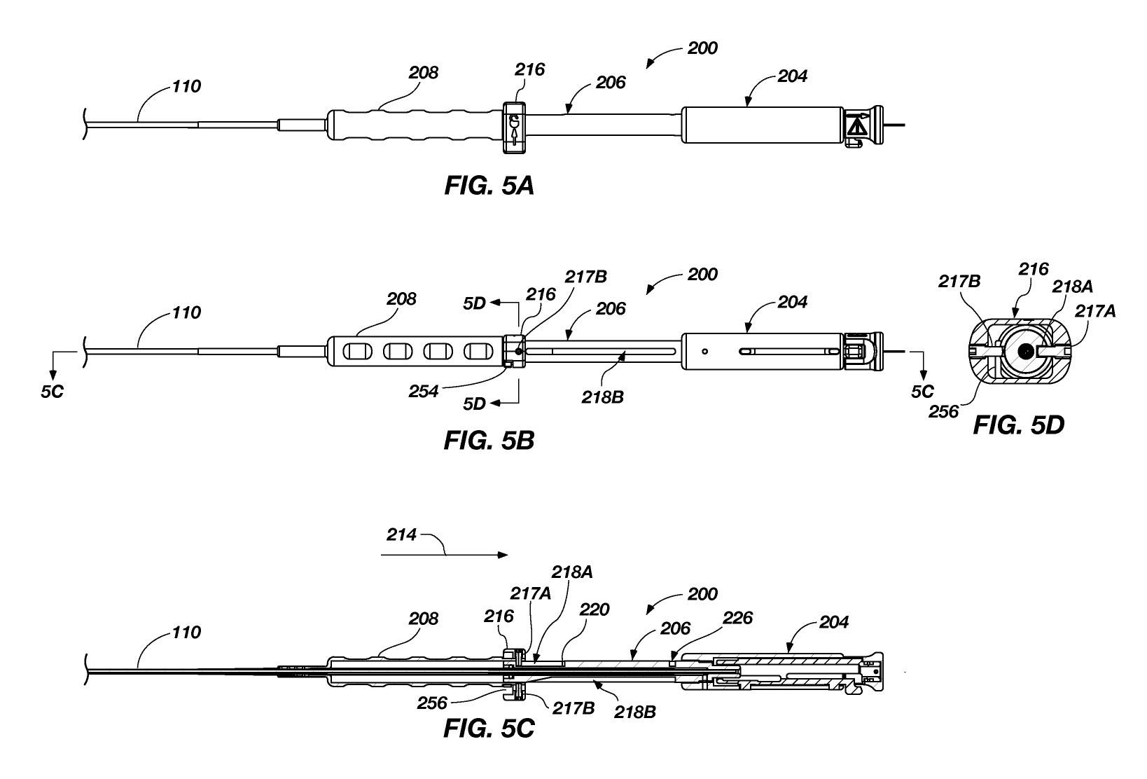

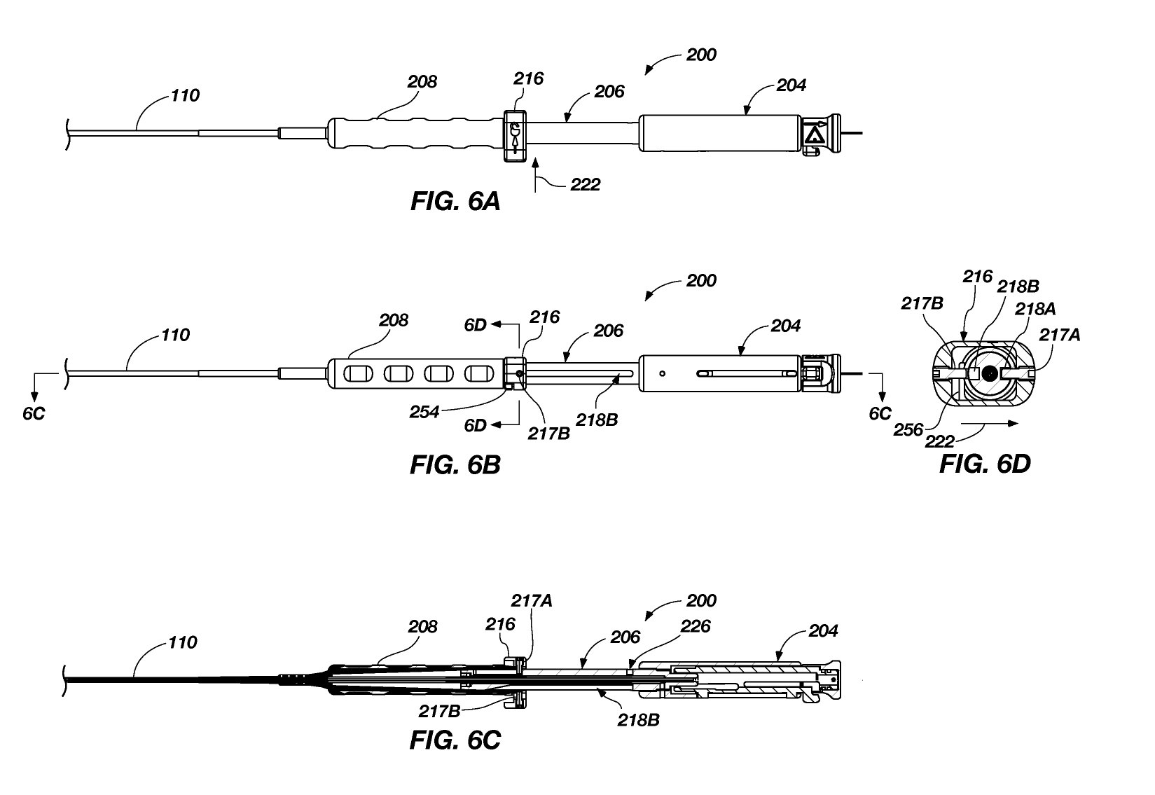

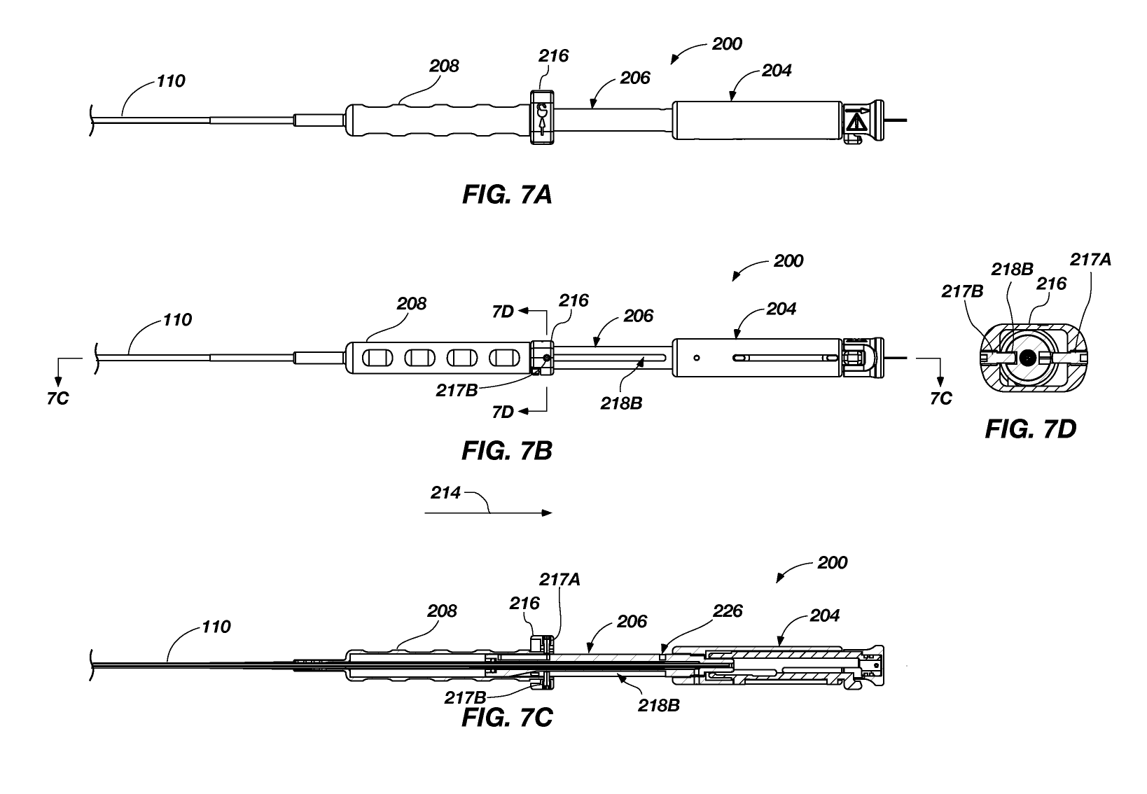

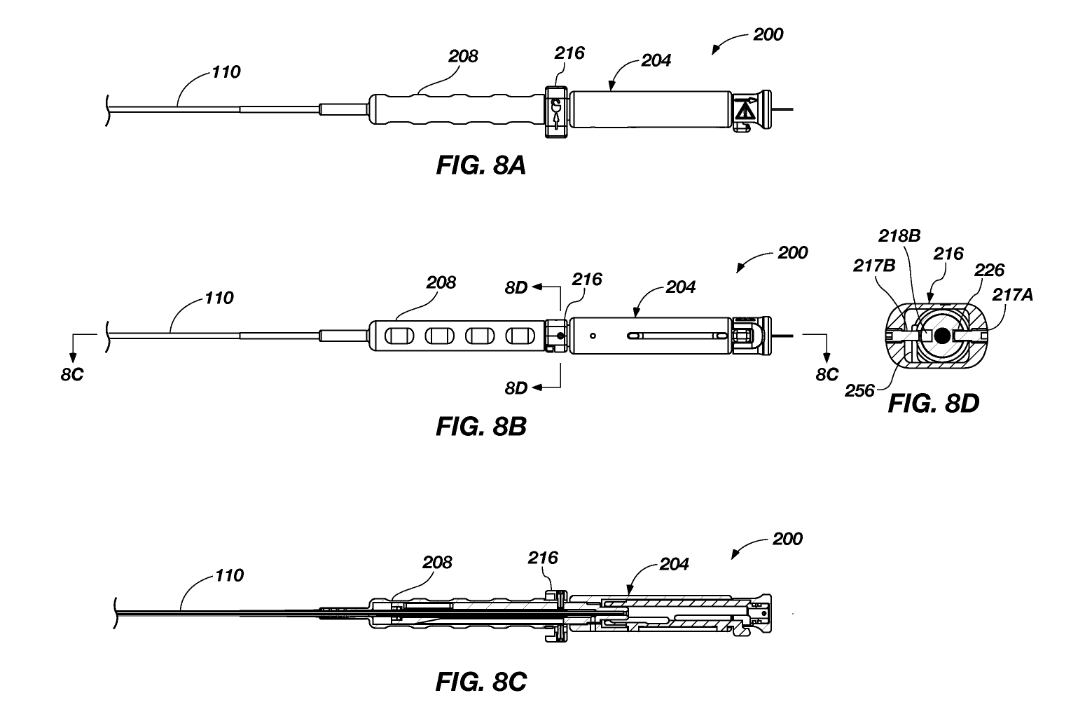

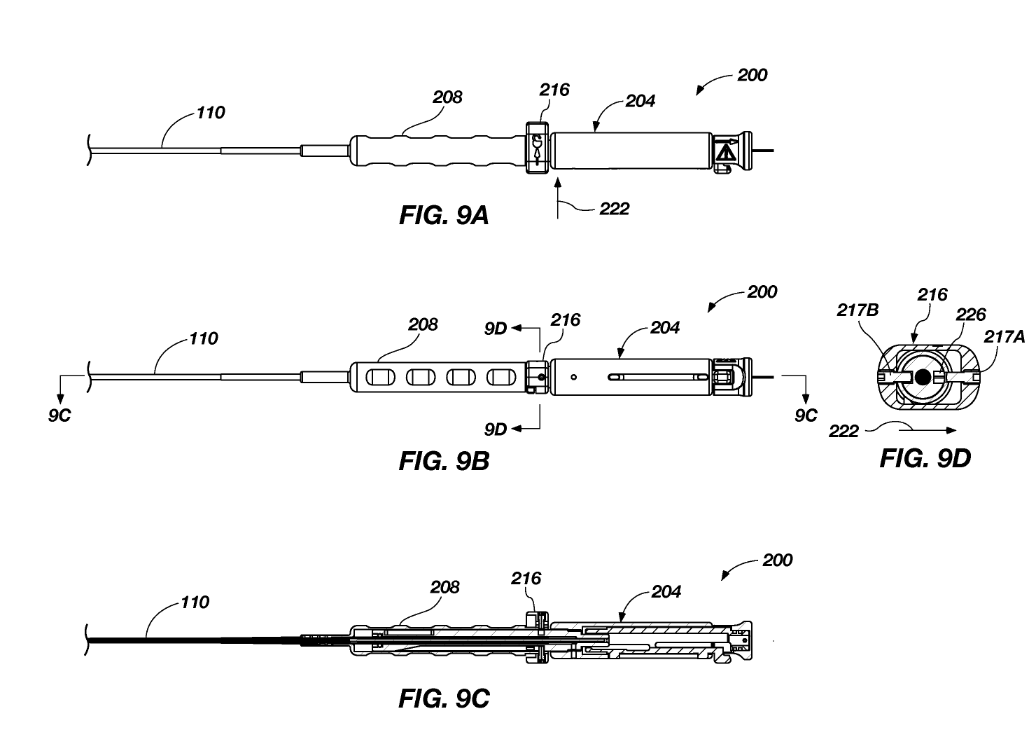

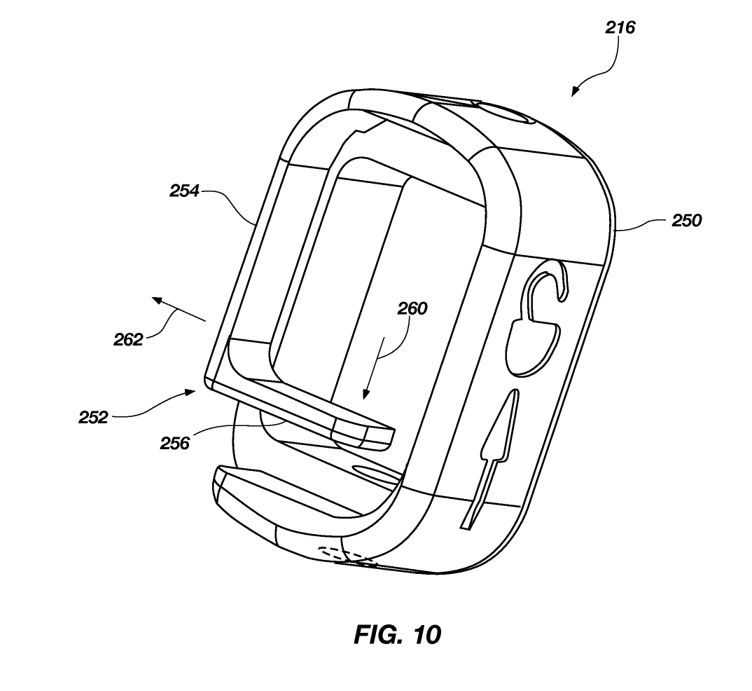

Referring now to FIGS. 5A-9D, another delivery system 200 is shown for delivering a medical device 102 to a desired location within a patient's body. It is noted that FIGS. 5A-5D are different views (as set forth in the Brief Description of the Drawings) of the delivery system 200 while in a first state, FIGS. 6A-6D are corresponding views of the delivery system 200 while in a second state, FIGS. 7A-7D are corresponding views of the delivery system 200 while in a third state, FIGS. 8A-8D are corresponding views of the delivery system 200 while in a fourth state, and FIGS. 9A-9D are corresponding views of the delivery system 200 while in a fifth state. It is noted that various components of the delivery system 200 are similar to the delivery system 100 described hereinabove. As such, certain components and elements share similar numbering as the elements shown in FIGS. 1A-4B for sake of convenience and clarity.

Referring first to FIGS. 5A-5D, the delivery system 200 includes a handle 204 having a handle extension 206. A slider or sleeve 208 is coupled with, and moveable relative to, the handle extension 206. A catheter 110 is coupled with the sleeve 208 and is displaceable in a corresponding manner with the sleeve 208 (i.e., when the sleeve 208 is displaced in a given direction, the catheter 110 is displaced with the sleeve 208 in the same direction). Although not expressly shown in FIGS. 5A-9D, the catheter 110 also includes a distal end 112 which houses the medical device 102 and from which the medical device 102 is deployed (such as is shown in FIGS. 1B, 2B, 3B and 4B).

When the delivery system 200 is in use, the sleeve 208 starts at an initial position which is also its distal-most position (i.e., furthest from the handle 204) such as shown in FIGS. 5A-5C. When in this initial position, an actuator 216, coupled with the sleeve 208, is in biased position wherein it is biased such that an associated first projection 217A (which may be configured, for example, as a set screw) is disposed within a first channel 218A of the handle extension 206 while a second projection 217B is not disposed in any channel or opening of the handle extension 208 but, rather, is positioned generally adjacent a surface of the sleeve 208.

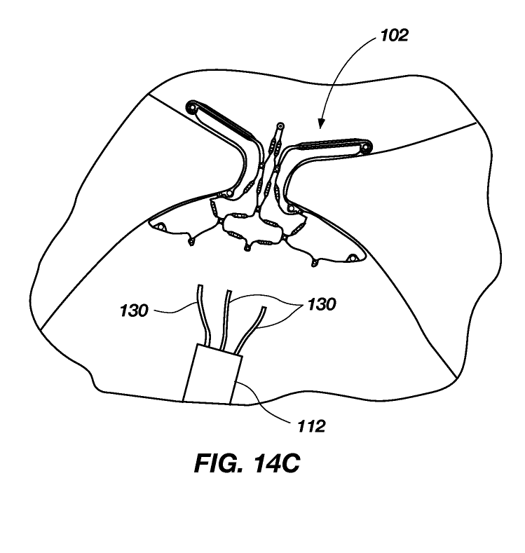

Once the catheter tip 112 is positioned at a desired location within a patient's body, the medical device 102 may be partially unsheathed by displacing the sleeve 208 proximally as indicated by directional arrow 214 to a first intermediate position as shown in FIGS. 6A-6D. The first channel 218A includes a shoulder or an abutment 220, against which the first projection 217A abuts, causing the sleeve 208 to stop at the desired intermediate position. When in this intermediate position, the medical device is partially unsheathed such as previously shown in FIG. 2B, or as also shown in FIG. 14A, by moving the catheter 110 proximally with the sleeve 208 relative to the medical device 102. It is noted that FIG. 14A shows the tip of the catheter 112 disposed through a Patent Foramen Ovale (PFO) 300 within a patient's heart between the right atrium 302 and left atrium 304, as will be appreciated by those of ordinary skill in the art and as described in considerable detail in U.S. patent application Ser. No. 11/836,123, previously incorporated by reference, and the medical device 102 partially unsheathed within the left atrium 304.

As seen in FIGS. 6A-6D (and primarily in FIGS. 6C and 6D), when the sleeve 208 is at the intermediate position with the first projection 217A abutting the shoulder or abutment 220 of the first channel 218A, the second projection 217B is now aligned with, but not yet disposed in, a second channel 218B. In the currently described embodiment, the second channel 218B is on an opposite side of the handle extension 206 relative to the first channel 218A while running parallel to the first channel 218A. Additionally, while the first channel 218A and the second channel 218B may be described as having their longitudinal extents overlapping, they are not coextensive. This is in contrast to the previously described embodiment (i.e., that which is described with respect to FIGS. 1A-4B), wherein the two channels 118A and 118B, while also being on opposing sides of their associated handle extension 106 and extending parallel to one another, where generally coextensive throughout their lengths, although a stopper 120 is positioned within the first or upper channel 118A.

With the medical device 102 partially unsheathed (see FIGS. 1B and 14A), the catheter 110 may be repositioned such that the distal portion 124 of the medical device 102 is anchored against the wall of the left atrium 304. Once the catheter tip 112 is properly positioned, the medical device 102 may be further unsheathed. In order to further unsheathe the medical device 102, the actuator 216 is displaced in a direction substantially different than the direction in which the sleeve 208 moves. In the present embodiment, the actuator 216 is displaced in a direction indicated by direction arrow 222 and which is substantially perpendicular to the direction indicated by directional arrow 214 in FIGS. 5A-5C. The actuator 216 is displaced, for example, by pressing the actuator in the indicated direction by one's fingers or hand with a force sufficient to overcome the biasing force provided by the actuator 216.

As shown in FIGS. 7A-7D, when the actuator 216 is displaced in the above-described manner, the first projection 217A is displaced out of the first channel 218A and the second projection 218B is displaced into the second channel 218B. This enables the sleeve 208 to be further displaced in the proximal direction as again indicated by directional arrow 214.

FIGS. 8A-8D show the delivery system 200 with the sleeve 208 displaced to its proximal-most position, wherein the medical device 102 is fully unsheathed as indicated in FIG. 4B and in FIG. 14B. FIG. 14B shows the medical device 102 positioned within a PFO of a patient's heart. As seen more particularly in FIGS. 8C and 8D, with the sleeve 208 at its proximal-most position, the actuator 216 is also displaced back to its normally biased position wherein the first projection 217A is disposed within an opening or aperture 226 formed in the handle extension 206 and the second projection 217B is displaced out of the second channel 218B. The positioning of the first projection 217A within the aperture 226 effectively locks the sleeve 208 from being displaced distally without the actuator 216 being affirmatively displaced or actuated so as to move the first projection 217A out of the aperture 226. This helps to protect against inadvertent displacement of the sleeve 208 relative to the handle 204 when a practitioner releases the medical device 102, such that the medical device 102 doesn't get repositioned or dislodged from it's intended placement prior to its release.

If, however, a practitioner decides that the medical device 102 is not properly positioned, or otherwise desires to reposition the medical device 102, the medical device 102 may be re-sheathed by displacing the actuator in the direction indicated by 222 to displace the first projection 217A from the aperture 226, as shown in FIGS. 9A-9D, and then displacing the sleeve 208 distally as indicated by directional arrow 228. It is noted that, in the presently described embodiment, there are no stopping members or structures to inhibit movement of the sleeve 208 between the proximal-most position of the sleeve 208 and the distal-most position of the sleeve 208 once the actuator 208 has been properly displaced. When the sleeve 208 reaches a longitudinal position such that the first projection 217A is aligned with the first channel 218A, the actuator 216 is biased back such that the first projection 217A is again positioned within the first channel 218A (as shown in FIGS. 5A-5D), thus, resetting the sheathing sequence.

Referring now to FIG. 10, an example of an actuator 216 is shown and described in further detail. The actuator 216 may be formed as a substantially unitary member having a body 250 configured to generally circumscribe the sleeve 208 (see FIGS. 5A-9D). A biasing member 252 is coupled with the body 250 and configured to bias the body 250 in a desired direction relative to the sleeve 208. In the example embodiment shown, the biasing member 252 may include a cantilevered, L-shaped leg having a first portion 254 with one end thereof coupled with the body 250 and a second portion 256 coupled with the first portion 254. In operation, the second portion 256 may be positioned to abut a surface of the sleeve 208. When a force is applied to the actuator 216 to effect displacement of the body 250 of the actuator 216 from its normally biased position, the biasing member 252 is displaced relative to the body 250 due to the interaction of the biasing member 252 with the sleeve 208. For example, the second portion 256 may be displaced in a direction as indicated by directional arrow 260. In another embodiment, the second portion 256 may be displaced in the first direction 260 as well as in a second direction as indicated by directional arrow 262. In such an embodiment, the first portion 254 (or at least the free end thereof) will also be displaced in the second direction 262. The biasing member 252 is formed of an elastic material and is designed such that, when the first projection 217A is aligned with either the first channel 218A or the aperture 226, the biasing member 252 will displace the body 252 and associated projections (not shown in FIG. 10) back to its normally biased state (i.e., the positions shown in FIGS. 5A-6D and 8A-8D).

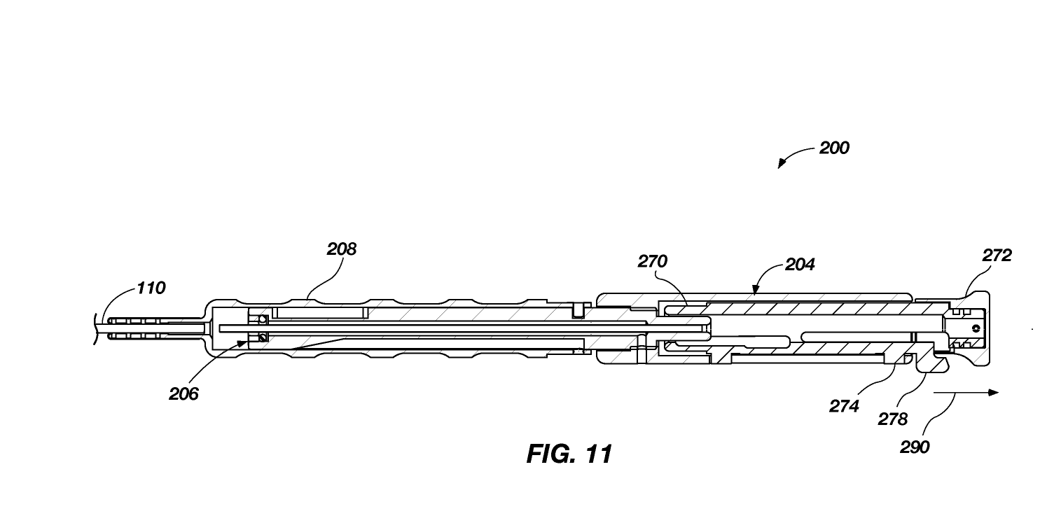

Referring now to FIGS. 11-13, additional details are shown of various components of the delivery system 200, including components associated with the release of the medical device 102 once it is unsheathed and satisfactorily positioned within a patient's body. FIG. 11 shows the handle 204, the handle extension 206, the sleeve 208 and the catheter 110. Partially disposed within the handle 204 is another component referred to herein as a release slider 270. The release slider 270 includes a projection or a knob 272 at the proximal-most end of the delivery system 200 that is exposed from the handle 204. The release slider 270 also includes a locking structure, such as a cantilevered projection 274 that engages the handle 204 and prevents the release slider 270 from being displaced relative to the handle 204 until the cantilevered projection 274 or other locking structure or mechanism is actuated.

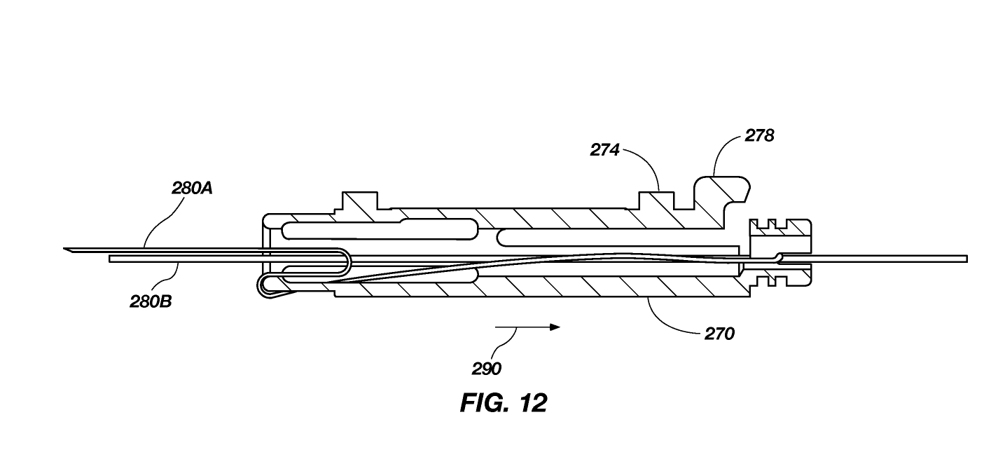

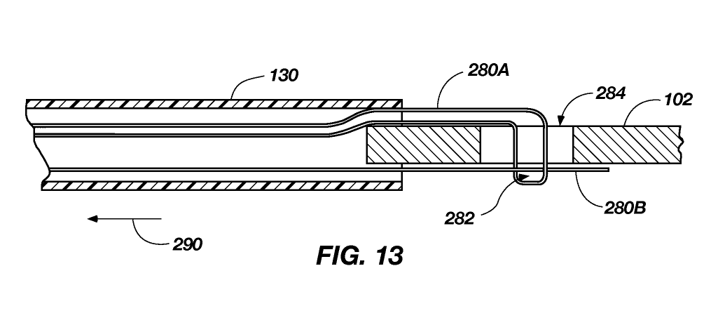

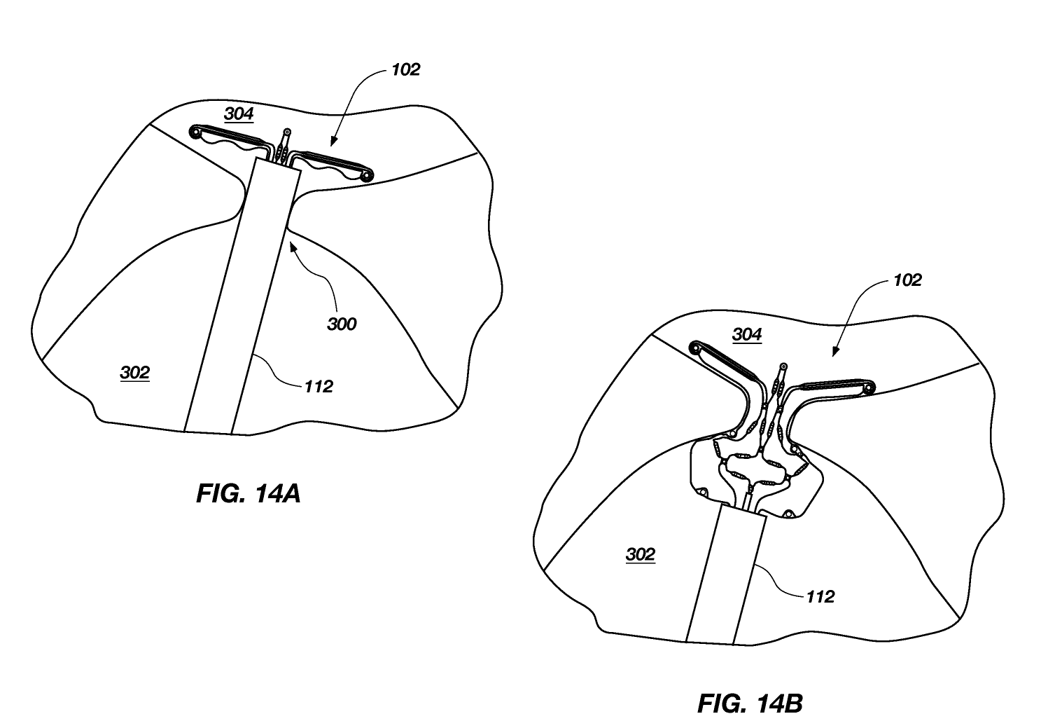

As seen in FIG. 12, the release slider 270 is also coupled to a plurality of wires or lines 280A and 280B, each of which is associated with a tether 130 (see FIGS. 4B and 14C). For purposes of clarity, only the lines associated with a single tether 130 are shown. FIG. 13 shows these same lines 280A and 280B coupled with a portion of the medical device 120. (It is noted that the view shown in FIG. 13 is reversed as compared to those shown in FIGS. 11 and 12, as indicated by directional arrow 290). For example, line 280A forms a loop 282 which extends through an opening 284 in the medical device 102 from a first side thereof. Line 280B extends through the loop 282 on a second side of the device and the loop 282 pulls against the second line 280B to retain the medical device until it is affirmatively released by a practitioner.

When it is desired to release the medical device 102 from the tethers 130, a button 278 may be depressed to disengage the cantilevered projection 274 from the handle 204 and the release slider 270 may be displaced in the direction indicated by directional arrow 290 by pulling on the knob 272. The displacement of the release slider 270 results in the displacement of the lines 280A and 280B. In displacing the lines 280A and 280B, one line 280B is configured to be initially taut, while the other line 280A is initially configured to exhibit a desired amount of slack. This results in the taut line 280B being withdrawn from the loop 282 while the slack is being removed from other line 280A. The other line 280A, once pulled taut, is subsequently displaced from the opening 284 of the medical device 102 and the medical device 102 is released from the tethers 130 as shown in FIG. 14C. The catheter 110 may then be removed from the patient with the medical device 102 securely in position within the patient's body. Other embodiments, including that which is described with respect to FIGS. 1A-4B, may include similar components for releasing the medical device 102.

As previously mentioned with respect to other embodiments, the components of the delivery system 200 may be formed of a variety of materials and utilizing a variety of manufacturing techniques as will be appreciated by those of ordinary skill in the art. Additionally, the delivery system 200 may be configured to unsheathe the medical device 102 in more stages than described in the example embodiment, or to unsheathe more or less or the medical device 102 at an intermediate stage. Also, various medical devices may be used and the specific embodiments are not to be considered limiting.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A delivery system for a medical device, comprising:
a handle;
a handle extension coupled with the handle,
a sleeve disposed about the handle extension;
a catheter coupled to the sleeve; and
an actuator associated with the sleeve;
wherein the sleeve is moveable from a first position to an second position to partially unsheathe a medical device from a distal tip of the catheter, and wherein, upon actuation of the actuator, the sleeve is moveable from the second position to a third position to fully unsheathe a medical device from the distal tip of the catheter.

2. The delivery system of claim 1, wherein the handle extension includes a first channel formed therein and at least one abutment surface associated with the first channel, wherein a first projection associated with the actuator abuts the at least one abutment surface to define the second position of the sleeve.

3. The delivery system of claim 2, wherein the actuator is displaced from a first actuator position to a second actuator position to disengage the first projection from the at least one abutment surface to enable the sleeve to be moved from the second position to the third position.

4. The delivery system of claim 3, wherein the actuator includes a biasing member configured to bias the first projection into the first channel when the first projection is aligned with the first channel.

5. The delivery system of claim 4, wherein the biasing member includes a cantilevered member coupled with a body of the actuator.

6. The delivery system of claim 5, wherein the cantilevered member includes an L-shaped member having at least a portion thereof configured to contact the sleeve.

7. The delivery system of claim 4, wherein the sleeve is moveable directly from the third position to the first position without actuating the actuator when the sleeve is proximate the second position.

8. The delivery system of claim 4, wherein the handle extension includes a second channel formed therein on an opposing side of the handle extension as compared to the first channel and extending substantially parallel to the first channel.

9. The delivery system of claim 8, wherein the first channel and the second channel are substantially coextensive in length.

10. The delivery system of claim 8, wherein the first channel and the second channel overlap one another in their longitudinal extents.

11. The delivery system of claim 8, wherein the actuator further includes a second projection, wherein, when the actuator is in the second actuator position, the second projection is disposed in the second channel.

12. The delivery system of claim 11, wherein, when actuator is in the first actuator position, the first projection is disposed within the first channel.

13. The delivery system of claim 11, wherein, when the sleeve is in the third position, and when the actuator is in the first actuator position, the first projection is disposed within an aperture so as to inhibit movement of the sleeve relative to the handle extension.

14. The delivery system of claim 11, wherein at least one of the first projection and the second projection comprise a set screw.

15. The delivery system of claim 1, further comprising a release mechanism coupled with, and moveable relative to, the handle, the release mechanism also being coupled to a plurality of lines configured for coupling with a medical device.

16. The delivery system of claim 15, wherein displacement of the release mechanism effects displacement of the plurality of lines.

17. A delivery system for a medical device, comprising:
a handle;
a handle extension coupled with the handle, the handle extension including at least one channel defined therein and along a longitudinal length of the handle extension and at least one abutment surface associated with the at least one channel;
a sleeve disposed about the handle extension;
a catheter coupled to the sleeve; and
an actuator associated with the sleeve and configured to be displaced between at least a first actuator position and a second actuator position, the actuator having at least one projection;
wherein the sleeve is moveable from a first position to a second position, wherein the at least one projection of the actuator abuts the at least one abutment surface and wherein the catheter is displaced to partially unsheathe a medical device from a distal tip of the catheter.

18. The delivery system of claim 17, wherein the actuator is displaceable from a first actuator position to a second actuator position to move the at least one projection out of engagement with the at least one abutment surface to enable the sleeve to move from the second position to a third position to fully unsheathe a medical device from the distal tip of the catheter.

19. A method of delivering a medical device, the method comprising:
housing a medical device within a tip portion of a catheter;
positioning the tip portion of the catheter at a desired location;
partially unsheathing the medical device by displacing a sleeve coupled with the catheter from a first position relative to a handle to a second position relative to the handle wherein, at the second position, an actuating assembly engages an abutment surface;
displacing the actuator to disengage the actuator from the abutment surface; and
displacing the sleeve and catheter to a third position relative to the handle to fully unsheathe the medical device.

20. The method according to claim 19, further comprising displacing the sleeve and catheter from the third position back to the first position to re-sheathe the medical device.

21. The method according to claim 19, further comprising releasing the medical device including displacing a release mechanism relative to the handle to displace a plurality of lines coupled between the release mechanism and the medical device.

22. The method according to claim 19, further comprising disposing the sleeve about a handle extension that is coupled with the handle and positioning a first projection of the actuator in a first channel of a handle extension when the sleeve is in the first position.

23. The method according to claim 22, further comprising disposing a second projection of the actuator in a second channel of the handle extension when the actuator is displaced for disengagement with the abutment surface.

24. The method according to claim 23, further comprising disposing the second projection in an aperture of the handle extension when the sleeve is in the third position.
